(19) United States
(12) Patent Application Publication
(10) Pub. No.: US 2007/0254051 A1
(43) Pub. Date: Nov. 1, 2007

(54) CHELATION WITH CONCENTRATED PLANT STEM CELL THERAPY

(76) Inventor: DOMINIQUE RICHARD, New York, NY (US)

Correspondence Address: NORRIS, MCLAUGHLIN & MARCUS, 875 THIRD AVE, 18TH FLOOR, NEW YORK, NY, 10022, US

(21) Appl. No.: 11/688,378

(22) Filed: Mar. 20, 2007

Related U.S. Application Data
(60) Provisional application No. 60/783,927, filed on Mar. 20, 2006.

(51) Int. Cl. A61K 36/87; A61K 36/76

(52) U.S. Cl. 424/725; 424/766; 424/769; 424/771

(57) ABSTRACT

A method of using a plant stem cell/plant growth hormone chelation preparation, including determining from medical testing the toxic condition of a patient, selecting a plant stem cell/plant growth hormone plant extract concentrate from buds of at least one plant having nutritional and medicinal constituents, such as from black poplar, grape vine, mountain pine, silver birch, white willow, European Elder. For oral administration of the plant stem cell/plant growth hormone plant extract is administered in an amount of about 3-15 drops, diluted in about half a glass of water, taken three times a day and continue for about 3 to 6 months or until a follow up testing provides a negative result. Depending on he state of toxicity, a combination of plant stem cell/plant growth hormone extracts may also be administered. A biologically active extract form at least two of the buds from the group of black poplar, grape vine, white willow, European elder and silver birch is also disclosed.

### BACKGROUND OF THE INVENTION

1. Field of the Invention

The present invention relates to a chelation method using concentrated Plant Stem Cells (PSC) and Plant Growth Hormones (PGH) for eliminating toxic substances from a human body.

2. Description of the Related Art

It is estimated that today about 70,000 chemicals are commercially used in the U.S. The EPA has classified 65,000 of such chemicals as potentially, if not definitely hazardous to human health. Despite constant efforts to contain such hazardous or toxic substances, they can be still be found around many work sites or in conjunction with many products, Many hazardous chemicals emanate into the environment, and thus may be found in the air, the soil and water supplies and are thus, easily absorbed by the human body. Toxic substances may also be found in connection with many products that by design come into close contacts with humans. They are found in food supplies and in cosmetic products and are used in dental care. Many of these toxins are known as heavy (toxic) metals i.e. metals having elemental densities above 7 g/cm³.

It is known that people exposed to hazardous substances physically suffer from such exposure in many different ways. It is also widely believed that conditions categorized as Autism Spectrum Disorders or Pervasive Developmental Disorders, Asperger's, PDD-NOS, ADD/ADHD, and many learning disabilities are based exposure to a hazardous chemical, specifically mercury.

It is also known that for example mercury toxins can impair the function of a person's immune system and as consequent cause chronic viral, bacterial and fungal illnesses. Both allopathic and holistic practitioners have observed that patients diagnosed with these illnesses, often have dramatic recoveries following an aggressive mercury detoxification program.

In order to eliminate hazardous chemicals or heavy metals from the human body various chelating agents are administered either orally or by intravenously. One of the known chelation agents is DMSA (meso 2,3 dimercaptosuccinic acid), DMSA is a dithiol, containing two sulfhydryl, or S—H groups, and an analogue of dimercaprol (BAL, British anti-lewisite), a lipid-soluble compound. DMSA is water soluble and is orally administered which creates a distinct advantage over BAL, which must be administered in an oil solution which results in a painful, deep intramuscular injection. DMSA, on the other hand, has a large therapeutic window and is the least toxic of the dithiol compounds. However, DMSA has been found to be potentially hepatotoxic. In addition, certain undesirable side effects are associated with the use of DMSA, such as bone marrow suppression and/or liver damage. Also, during therapy with DMSA, neutropenia and thrombocytopenia have been reported. DMSA can cause deficiencies of copper, manganese, molybdenum and zinc, if they are not replaced by supplementation. DMSA doesn't directly bind magnesium, cysteine, or glutathione, but heavy metal detoxification can result in depletion of these nutrients as well.

DMPS (2,3-dimercapto-1-propanesulfonic acid sodium), is also used as a chelating agent, however, there are reports that DMPS also binds and therefore removes beneficial minerals from the body. Compared to DMPS is considered less safe that is DMPS proved to be three times more toxic than DMSA and DMSA is much less likely to bind beneficial minerals.

There is yet another chelation agent for the removal of toxic or heavy metals from the human body, which is EDTA (calcium ethylenediamine tetra-acetic acid). EDTA may be administered either by a series of intravenous infusions or by oral route and even rectal suppositories. Unfortunately, EDTA, like DMSA and DMPS also removes essential minerals from the body, making EDTA less than ideal, especially for administering the compound to children.

These known chelation methods may be administered either orally or intravenously. Oral administration of chelating agents has the drawback that it puts stress on an already stressed gastrointestinal system and that some portion of the chelator is absorbed in the stomach and never makes it into the bloodstream where it can then bind to the toxins for elimination.

Thus, with all discussed know chelation methods, two dangers have been observed, which are (1) possible liver damage and (2) extreme trace mineral depletion.

Recently, a new chelating method was proposed by the University of Pennsylvania. This method is based on enzyme phytochelatin. Phytochelatin utilizes the polymerizing of glutathione, a sulfur-rich peptide with a high affinity for toxic metals. The products of these reactions, phytochelatins, bind very strongly onto heavy metal atoms, immobilizing them and preventing them from moving to parts of the cell where their toxic effects are exerted. However, since this is a relatively recent study, not much is known about the success and the existence of any side effect.

Yet another study emerged which is based on therapeutic doses of Cilantro. It was observed that Cilantro rapidly mobilized mercury and other toxic metals from the brain. Cilantro acts as a reducing agent changing the charge on the intracellular mercury to a neutral state, allowing the mercury to diffuse down its concentration gradient into connective tissue. This is called connective tissue mercury toxicity.

It is also known that phytoremediation are successfully utilized to clean up metals, pesticides, solvents, explosives, crude oil, polyaromatic hydrocarbons and landfills with great success. For example, hybrid poplar and Eastern cottonwood remove chlorinated solvents in ground water. Petroleum and its hydrocarbons can be removed from soil and ground water using alfalfa, poplar and juniper, fescue grass, crabgrass, and clover. Polyaromatic hydrocarbons are remediated with ryegrass and mulberry trees. Heavy metals can be removed from soil using poplar and pine trees, vines, willows, chaparral, various grasses, and castor plants. Radionuclides can be removed from ground water with sunflowers and water hyacinth, and from the soil with mustards and cabbage. Explosives such as TNT can be removed from groundwater with duckweed and parrot feather grass. Nitrates can be remediated with cottonwood and poplar trees. Rye removes polyaromatic hydrocarbons. Various water plants, including hyacinths, are being used in municipal sewage treatment.

Phytoremediation can be categorized into six basic plant functions:

(1) phytodegradation, (2) phytoextraction, (3) rhizofiltration, (4) rhizodegradation, (5) phytostabilization, and (6) phytovolatilization. These functions are clear examples of the eco-physiology of plants and its practical applications for environmental remediation. Several comparisons can be made between these plant processes and human metabolic functions.

### Medicinal Plants Used in Eco-Restoration

Several plants with important nutritional and medicinal properties are being utilized in ecological restoration and environmental remediation. These species represent a unique category of phytoremeditation and plant eco-physiology: plants, which benefit the environment while simultaneously providing food and medicine.

1,4-dioxane, a suspected carcinogen, is widely used as a solvent in paints, varnishes, lacquers, cosmetics, and deodorants. It exists as a liquid at room temperature, is fully miscible in water, and is expected to be highly mobile in soil. Its half-life in soils and ground water is on the order of years, while its half-life in the atmosphere in the presence of NO and hydroxyl radicals is only 6.7-9.6 hours. Therefore, 1,4-dioxane volatilization into the atmosphere by plant transpiration could be a desirable result.

In this research we assessed the capacity of hybrid poplar trees (Populus deltoides nigra, DN34, Imperial Carolina) for uptake and translocation of 1,4-dioxane using I4C-labeled dioxane in hydroponics experiments. Plants can enhance the removal of xenobiotics by at least two mechanisms: (1) direct uptake and, in some cases, in-plant transformations to less toxic metabolites; and (2) stimulation of microbial activity and biochemical transformations in the root zone through the release of exudates and enzymes. Advantages of hybrid poplar trees as phytoremediation tools include:

1,4-dioxane was readily taken-up by the hybrid poplar tree cuttings from hydroponics solution. After 8 days, the following results were obtained:
30-79% (average=54%) of the dioxane mass had been removed from the planted reactors
10% removed from the excised tree reactors
8% removed from the unplanted control
Concentration of 1,4-dioxane remained relatively constant in all reactors, indicating that the compound may be freely diffusing into the plant via water osmosis.

Rapid uptake of 1,4-dioxane by hybrid poplar trees makes phytoremediation appear as an attractive alternative at dioxane-contaminated sites. Further research will examine poplar removal of 1,4-dioxane from contaminated soil.

Glutathione (GSH) and its derivatives play the major role in plant defense against these pollutants. Pesticides are detoxified by conjugation with GSH by glutathione S-transferase and subsequent excretion of these conjugates in the vacuoles. Heavy metals induce synthesis of a wide range of cysteine-rich peptides and proteins, including metallothioneins and phytochelatins (PC). The latter are synthesized enzymatically from glutathione, bind the metals with high affinity and the PC-metal complex is sequestered to the vacuole.

### SUMMARY OF THE INVENTION

The present invention eliminates the shortcomings known in connection with the prior art chelation agents. One aspect of the invention is to provide a new method of chelating with the new agent. This new chelation agent is based on using concentrated plant stem cells and it overcomes the disadvantaged associated with the known chelation agents. Depending on which specific toxins need to be removed from the body, plant stem cells from different plants are used. Presently, plant stem cells/plant growth hormones form six different plants have been used in chelation agents. The advantage of chelation with plant stem cells/plant growth hormones is that this method does not remove essential minerals.

In addition, plant stem cell/plant growth hormone-based chelation, if administered orally, puts no stress on the already stressed gastrointestinal system. The plant stem cell/plant growth hormones based agent more easily reaches the bloodstream through osmosis where it binds to the toxins for elimination. In addition, it was observed that with plant stem cell chelation, liver, kidneys and brain cognitive functions were greatly restored.

Chelation with plant stem cells (phytochemicals constituents) belongs to the field of phytotherapy. Understanding bioavailability is the key to assessment of the potential toxicity of metals and their compounds. Bioavailability depends on biological parameters and on the physicochemical properties of metals, their ions, and their compounds. These parameters, in turn, depend upon the known atomic structure of the metals.

The bud concentrate (or young shoots or rootlets) concentrate is prepared through a maceration process using water, alcohol and glycerine and bottles until used by a patient. The growers, botanists and chemist insist on water for mapping purposes of oligo-elements extraction.

An earlier process underwent an initial maceration process which used frozen buds which lose 10% of their effectiveness. The buds are frozen so they can be macerated as needed for the thousands of extracts produced in homeopathic laboratories. PSC/PGH therapy is most effective when it comes from freshly macerated buds. The 1DH dilution came about as part of an attempt to make a PSC/PGH therapy product into a homeopathic product, which it is not. Also the amount of drops needed and the amount of alcohol makes it less desirable for an already burden body and defeats the chelation process for proper detoxification.

Concentrated plant stem cells and plant growth hormones are derived from the embryonic stage of certain plants. The embryonic stage are buds and young shoots and young roots. In such buds, shoots or roots, the plant growth hormones Auxins and Gibberellins are present. These Auxins and Gibberellins are capable of repairing the ribonucleic acid (RNA) also they cause tissue excitation which helps in the release of toxins which are embedded in the tissue. The embryonic plants also contain Cytokinin, Abscisic Acid and Ethylene and Meristem. The Meristems are the plant stem cells (PSC). Also present in the embryonic stage are vitamins, enzymes phytochelatin synthase, oligo-elements, tannins, flavanoids, phenols, quercetin, myricetine, nucleic acids, and antioxidants. PGH/PSC are important active parts of a plant because they contains all of the genetic information of the future plant. Once the plant matures, PSC/PGH are no longer present. When administered to humans, the PSC/PGH have the capacity to detoxify, oxygenate, nourish and rejuvenate the cells. The rejuvenation is caused by the auxin, which contains an indole-acetic acid, which also provides an anti-inflammatory effect. Similar to human stem cells, the plant stem cells regulate the function of organs and glands in the human body. They stimulate the reticuloendothelial system, the emunctory functions and facilitate detoxification of the organism. Plant stem cells also affect cellular ageing with the only exception of the genetic code. The plant stem cells also repair the damage to the genetic transcription.

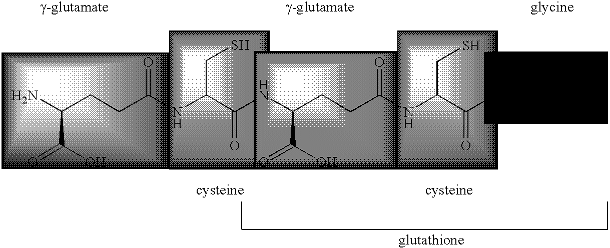

Phytochelatins (PCs) is a technology also known as botanical metal hyperaccumulation. Phytochelatins Roles in Heavy Metal Detoxification and Homeostasis. Phytochelatins consist of just three amino acids; Cysteine, Glycine, and Glutamic acid, arranged generally in a (y-GluCys) n-Gly conformation. This conformation proves to be significant in the identification of the origin of PCs. The fact that PCs are arranged in a y-carboxylamide bond suggests that the phytochelatins are not a direct result of expression of a metal tolerance gene, but rather a product of a biosynthetic pathway, with glutathione, a detoxifying agent, most likely the substrate on which the pathway begins.

The Biosynthesis of PCs can be broken down into a very basic model, which is useful in deciphering specific models of known pathways. The synthesis of PCs is a response to the addition of heavy metals, so with the application of a heavy metal to a plant, PC synthesis occurs. The heavy metal activates the enzyme phytochelatin synthase, which acts upon a glutathione substrate to produce PCs. (Murphy and Taiz, 1995). This action continues until all of the metal is complexed. With this basic model in place, one can understand a specific pathway of the biosynthesis of PCs. Figure below shows the mechanism for plants exposed to elevated environmental levels of heavy metals, which is the most effective elicitor of PCs. When the cell detects heavy metals, Glutamine and Cysteine molecules are changed by the enzyme y-glutamylcysteine synthase, coded for by the CAD2 gene, with an end product of y-GluCys. The y-GluCys is then transformed into glutathione (GSH) by the enzyme glutathione synthase. Once at the stage of Glutathione, two steps can occur: the first is the binding of two GSH molecules to a Heavy Metal molecule and the passage of this compound into a storage vacuole; the other option is that the GSH is acted upon by the enzyme phytochelatin synthase, coded for by the gene Cad1, resulting in functional phytochelatins. These proteins then bind with metal ions and create low molecular weight PC-Cd complexes, which pass into a storage vacuole, and react with sulfides to make high molecular weight CdS-PC complexes. (Raskin. 1995)

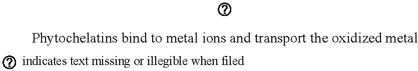

### Detoxification of Arsenic by Phytochelatins in Plants

As is a ubiquitous element present in the atmosphere as well as in the aquatic and terrestrial environments. Arsenite and arsenate are the major forms of As intoxication, and these anions are readily taken up by plants. Both anions efficiently induce the biosynthesis of phytochelatins (PCs)

In order to understand chelation with concentrated plant stem cells, it is important to understand the type of toxic substance that is targeted for elimination and how the body eliminates different types of toxic substances.

Toxins accumulated in the human body may be classified as (1) exogenous, (2) endogenous and (3) autogenous:

(1) Exogenous toxins come from external sources such as tobacco, drugs, stimulants and amalgam fillings. Atmospheric pollutants such as motor exhaust, carbon dioxide, lead, nitrogen dioxide and sulphur dioxide are exogenous toxins. Also included in this category are mental and emotional factors capable of interfering with normal bodily processes, such as anxiety, grief, depression, stress, worry, emotional relationships and so on.

(2) Endogenous toxins are the result of viral or bacterial infections affecting the normal functions of the body and mycostatic (mold preventing) activity. This can include the by-products of the waste metabolism of yeasts, molds and fungus.

(3) Autogenous toxins are generated within the body from heredity weakness such as miasmatic influences such as psora, sycosis, etc.

Waste is excreted from the body by that emunctory organ which is specially adapted to the work that function, for example, the kidneys eliminate urea and the lungs eliminate carbon dioxide. Neither of these organs is so constituted that it can do the work of the other. Hence, when the blood passes through the lungs, only carbon dioxide is eliminated and not urea. Similarly, when blood passes through the kidneys only urea is removed and not carbon dioxide. When a patient takes certain drugs, they are expelled in a specific way, one drug is expelled by vomiting, another by diarrhea, another by diuresis, another by diaphoresis and still another by expectoration. Other substances, not easily eliminated through these channels, are sent out through the skin in the form of skin eruptions. Each organ appears to excrete the drug that it can handle best. Accordingly, substances to be eliminated are categorized as either emetic, purgatives, diaphoretics or diuretics:

Emetics do not act on the stomach, but are rejected by the stomach. Purgatives do not act on the bowels, but are expelled through the bowels. Diaphoretics, instead of acting on the skin, are eliminated through the skin. Diuretics do not act on the kidneys, but they are eliminated through the emunctory.

Thus, one may speak of selective elimination: All injurious substances, which gain access into the domain of vitality, are counteracted, neutralized and eliminated in such a manner and through such channels as will produce the least amount of wear and tear to the organism.

It is important to recognize various phases of elimination of toxins:

The first three phases are called humoral phases because intra-cellular systems are for the most part not disturbed. The defense systems of the body are intact and capable of responding to homotoxins by eliminating them through various body orifices (including the skin and lungs). These phases are called the excretion, reaction and deposition phases: The excretion phase eliminates toxins through the orifices. During the reaction phase inflammation is the primary means the body utilizes to remove homotoxins. During the deposition phase toxins are both deactivated and stored.

As discussed above, PSC/PGH have the capacity to detoxify, but also oxygenate, nourish and rejuvenate the cells. The rejuvenation is caused by the auxin, which contains an indole-acetic acid, which also provides an anti-inflammatory effect. The PSC/PGH regulate the function of organs and glands in the human body. They stimulate the reticuloendothelial system, the emunctory functions and facilitate detoxification of the organism. PSC/PGH also effect to a certain degree cellular aging.

Observed over a period of at the last 10 years, all patients from pediatric to geriatric population underwent a 24 hour-urine-test (collecting urine over a period of 24 hours and have a laboratory analysis done) or a hair analysis to determine the toxins present. Which ever test is performed and turn out positive, i.e. a toxin were present, the patient was given a PSC/PGH therapy agent and within 3-6 months, the patient was clear of heavy metals and tested negative. However, the most surprising result was that symptoms associated with metal toxicity, for example, from cognitive to neurological afflictions and symptoms associated with muscular stress had greatly improved. None of the side effects such as essential mineral depletion was ever observed nor was the liver stressed by this method, contrary to the use of EDTA, DMPS, and DMSA.

Another advantage is that since PSC/PGH agents may be orally administered, they do not have the invasiveness of the intravenously administered prior art chelation agent. Therefore, the PSC/PGH therapies are extremely well suited for the pediatric population, who may not tolerate the trauma and discomfort of intravenous chelation.

An additional advantage over the prior art chelation is that PSC/PGH therapies are considerably less expensive than the prior art chelation agents.

Yet another advantage is that a PSC/PGH therapy eliminates the toxins considerably faster that a chelation therapy with EDTA. EDTA treatments extend generally to about 2 years.

There are frequent reasons, resulting from examining patients and testing them to determine the presence and type of toxicity, when a single source of PSC/PGH agent is not sufficient and it becomes necessary to build a complex therapeutic strategy for several sources of PSC/PGH agents. A PSC/PGH agent, originating from one plant is used when a single toxic metal was found in the patient, and a combination of agents when more than one is found.

Obviously, there are many factors cooperating in the development of the struggle against eliminating toxic substances form the patient. Therefore, in the PSC/PGH therapeutic strategy it will be normal to prescribe a combination of agents, because in combination, many synergistic effects have been observed in obtaining corrective results. Approaching one problem at a time will not achieve the overall balance of the entire organism. It is often not enough to eliminate toxic accumulation at the cellular level but it also is necessary to stimulate the proper function of organs in order to improve the patient's overall health after being under the toxic stress for a period of time.

The following table demonstrates the type of toxins most commonly found in patients and the source of such toxins and the symptoms exhibited by most patients:

| METAL | | |
|---|---|---|
| TOXICITY | SOURCES | SYMPTOMS |
| Aluminum | Today, aluminum is everywhere - under your arms | Elevated aluminum has been implicated for years in |
| | (deodorants), in your teeth (toothpaste), and on your baby's | several brain diseases, such as Alzheimer's and |
| | skin (powder). In addition, dental amalgams, many cosmetics, | Parkinson's disease and also found in some seniors |
| | and cigarette filters contain aluminum. We ingest in some | with extreme memory loss, absent-mindedness, or |
| | drinking waters, commercial teas, cheeses, white flour, baking | dementia. It is sometimes found in the hair of children |
| | powder, aspirin, and table salt. We cook with it too; most pots | diagnosed with ADHD, ADD, and those with seizures. |
| | and pans are at least in part, made of aluminum. | Hyperactivity, memory disturbances, and learning |
| | Unfortunately, many over-the-counter and prescription | disabilities many result from even mildly elevated levels |
| | antacids for digestive difficulties, contain aluminum. Aluminum | of aluminum. Inhibition of neurotransmission and |
| | may also leach out of aluminum foil or cans into food and | impaired motor coordination may also result. According |
| | beverages. Sodas (with phosphoric acid), tomato sauce, | to a report in Lancet in 1989, many infant formulas |
| | pineapple, and coffee in aluminum cans are major culprits, as | contain aluminum. In this report it was revealed that |
| | well as food wrapped in aluminum foil. Commercial tomato | human breast milk contained 5–20 micrograms per liter |
| | sauces are often prepared in huge aluminum pots and the | of aluminum, cow's milk-based formulas contained 20 |
| | acidity of the tomatoes can cause the leaching of aluminum | times as much aluminum, and soy-based formulas |
| | from the cookware into the finished product. Coffee prepared | contained 100 times as much. So human breast milk |
| | in aluminum pots and pans may also be toxic. Heavy coffee | had the lowest concentrations, proving to be the safest. |
| | drinkers may also be at risk another way. It is speculated that | Physical symptoms of aluminum toxicity may include |
| | because coffee drinking causes an acidic reaction in the | brittle bones or osteoporosis, as aluminum is stored in |
| | digestive tract, aluminum contained in any food or drug may | the bones. Kidney malfunction may also result, as the |
| | become toxic or more toxic, because the acid will cause it to | kidneys filter aluminum. |
| | collect in the bloodstream. There it can be carried to the brain | |
| | along with the very blood that's supposed nourish the brain | |
| | cells. | |
| Arsenic | Arsenic toxicity includes ingestion of arsenic (found insect | Nausea or vomiting, abdominal pain, diarrhea, |
| | poisons), skin contact (e.g. some linseed oils) and even | headache, vertigo, fatigue, parasthesia, paralysis and |
| | drinking water. | mental impairment. Allergies, hair loss, colic. |
| Cadmium | The highest contributor to cadmium toxicity is cigarette | Blood pressure problems, emphysema, kidney |
| | smoke; it is found in cured tobacco. This is toxic for both the | problems, prostate and other cancers, joint pain. |
| | smoker and the non-smoker. First hand and second hand | Cadmium may promote skeletal demineralization and |
| | smoke are high sources of cadmium. Other sources are well | increase bone fragility and fracture risk. Cadmium can |
| | water, some soft water, evaporated milk, and some organ | weaken the immune system and allow bacteria, |
| | meats such as kidney and liver. Cadmium pipes can be a | viruses, yeast, and parasites to proliferate. Cadmium |
| | source, as well as fungicides sprayed on apples, tobacco, and | may also promote skeletal demineralization and |
| | potatoes. | increase bone fragility and fracture risk. Symptoms of |
| | | cadmium toxicity include sore joints, decreased |
| | | appetite, slow growth, zinc deficiency and kidney |
| | | stones. |
| Copper | Beer, chocolate, copper cookware, copper IUD's, copper | Migraine, emotional problems including depression, |
| | pipes, dental prosthesis, fungicides, hemodialysis, ice | hyperactivity and schizophrenia (histapenia type), liver |
| | makers, industrial emissions, industrial wastes, | disease. |
| | insecticides, liver, | |
| | milk, nuts, oysters, swimming pools, | |
| | water, city water, well water. | |
| Lead | Human exposure to lead occurs primarily through drinking | Learning problems, emotional disturbances, |
| | water, airborne lead-containing particulates and lead-based | nervousness, anemia, anxiety, loss of appetite, |
| | paints. Several industrial processes create lead dust/fumes | headache, convulsions, hair loss, epilepsy. |
| | resulting in its presence in the air. Mining smelting and | hypertension, fatigue, hemolytic anemia, abdominal |
| | manufacturing processes the burning of fossil fuels | pain, nausea, constipation, weight loss, peripheral |
| | (especially lead-based gasoline) and municipal waste and | neuropathy, cognitive dysfunction, arthralgia (joint |
| | incorrect removal of lead-based paint results in airborne lead | pains), headache, weakness, irritability, impotence, |
| | concentrations. After lead is airborne for a period of ten days | loss of libido, depression, decreased thyroid and |
| | it falls to the ground and becomes distributed in soils and | adrenal function, chronic renal failure, gout. High lead |
| | water sources (fresh and salt water surface and well water | levels have been found to be associated with ADHD, |
| | and drinking water). However the primary source of lead in | impulsivity, and an inability to inhibit inappropriate |
| | drinking water is from lead-based plumbing materials. The | responses. Hypertension, fatigue, hemolytic anemia, |
| | corrosion of such materials will lead to increased | abdominal pain, nausea, constipation, weight loss, |
| | concentrations of lead in municipal drinking water. Lead from | peripheral neuropathy, cognitive dysfunction, |
| | water and airborne sources have been shown to accumulate | arthralgia, headache, weakness, irritability, impotence, |
| | in agricultural areas leading to increased concentrations in | loss of libido, depression, depression of thyroid and |
| | agricultural produce and farm animals. Cigarette smoke is | adrenal function, chronic renal failure, gout. |
| | also a significant source of lead exposure; people who smoke | |
| | tobacco or breathe in tobacco smoke may be exposed to | |
| | higher levels of lead than people whom are not exposed to | |
| | cigarette smoke. | |
| Manganese | Manganese overload is generally due to industrial pollution. | Nervous disorders. Symptoms of toxicity mimic those |
| | Workers in the manganese processing industry are most at | of Parkinson's disease (tremors, stiff muscles) and |
| | risk. Well water rich in manganese can be the cause of | excessive manganese intake can cause hypertension |
| | excessive manganese intake and can increase bacterial | in patients older than 40. Significant rises in |
| | growth in water. Manganese poisoning has been found | manganese concentrations have been found in |
| | among workers in the battery manufacturing industry. | patients with severe hepatitis and post hepatic |
| | Everyone is exposed to small amounts of manganese in air, | cirrhosis, in dialysis patients and in patients suffering |
| | water, and food. Exposure to metal fumes from welding, | heart attacks, Manganese has been known to cause |
| | cutting and brazing-especially in confined spaces-can cause | the degenerative brain disorder known as |
| | brain damage. A major culprit is manganese, a component of | Parkinsonism since 1837. A recent study found 40 |
| | all steel and major welding materials. | percent of welders showed signs of the disorder. |
| Selenium | Selenium is commonly found in rocks and soil. In the | Nervousness, vomiting, cough, dyspnea, convulsions, |
| | environment, selenium is not often in the pure form. | abdominal pain, diarrhea, somnolence, fall in blood |
| | Much of the selenium in rocks is combined with sulfide | pressure, respiratory failure and death. Garlic-like odor |
| | minerals or with silver, copper, lead, and nickel minerals. | may be present on breath. Selenium oxychloride is a |
| | Selenium and oxygen combine to form several compounds. | severe vesicant and may cause fatal poisoning. |
| | Selenium sulfide is a bright red-yellow powder used in anti- | Accidentally swallowing a large amount of selenium |
| | dandruff shampoo. Industrially produced hydrogen selenide is | (e.g. a very large quantity of selenium supplement pills) |
| | a colorless gas with a disagreeable odor and is probably the | could be life threatening without immediate medical |
| | only selenium compound that might pose a health concern in | treatment. Chronic toxicity: marked pallor, garlic odor |
| | the workplace. Selenium dioxide is an industrially produced | or breath, sweat and urine; red staining of fingers, |
| | compound that dissolves in water to form selenious acid. | teeth and hair, marked debility, depression, epistaxis, |
| | Selenious acid is found in gun blueing (a solution used to | G.I. disturbances, dermatitis, irritation of nose and |
| | clean the metal parts of a gun). Selenium is used as | throat. Hydrogen selenide can cause pneumonitis and |
| | ingredient in toning baths in photography; as pigment in | damage to liver and spleen. People may also lose |
| | manufacturing ruby-, pink, orange, or red-colored glass; as | feeling and control in the arms and legs. |
| | metallic base in making electrodes for arc lights, electrical | |
| | instruments and apparatus, selenium cells, telephotographic | |
| | apparatus; as coating in flame proofing electric cables; as | |
| | vulcanizing agent in processing of rubber; as catalyst in | |
| | determination of nitrogen by kjeldahl method; for | |
| | dehydrogenation of organic compounds. Although selenium | |
| | can be toxic by itself, it also prevents the toxicity of several | |
| | other metals such as silver, mercury, cadmium, and lead. | |
| | Mercury causes the loss of the needed metals copper and | |
| | zinc, and selenium helps to prevent that loss by binding the | |
| | mercury. | |
| Mercury | The source of mercury poisoning is primarily contamination of | The onset time is one week or more, at which time the |
| | food by polluted water containing mercuric compounds from | symptoms of numbness, weakness of the legs, spastic |
| | industrial waste or organic mercury contained in some | paralysis and impaired vision are noted. Blindness and |
| | fungicides. Food or feed grains treated with mercury- | coma are extreme symptoms of the poisoning. |
| | containing fungicides are a potential source for transmission | Nervous and digestive disorders, allergies, tremors, |
| | of the metal through both animal and cereal foods. Chronic | infertility, depression, psychosis, asthma, infertility. |
| | mercury exposure and toxicity can be very general and | |
| | difficult to diagnose based on symptoms alone. In addition, | |
| | individuals show varying levels of sensitivity to the presence | |
| | of mercury: amalgam removal may be very important in the | |
| | recovery process of one person, while for another it may be | |
| | best to leave the amalgams in place. | |
| Iron | Discussion of iron toxicity in this protocol is limited to ingested | |
| | or environmental exposure. Iron does not appear on the | |
| | ASTDR'S “TOP 20 List,” but it is a potential toxic metal of | |
| | concern, particularly because ingesting dietary iron | |
| | supplements may acutely poison young children (e.g., as few | |
| | as five to nine 30-mg iron tablets for a 30-lb child). Other | |
| | sources of iron are drinking water, iron pipes, and cookware. | |
| | Target organs are the liver, cardiovascular system, and | |
| | kidneys (Roberts 1999). | |
| Nickel | Nickel is a very abundant element. In the environment, it is | Allergies, skin problems such as dermatitis, dizziness, |
| | found primarily combined with oxygen (oxides) or sulfur | digestive disorders, cancer esp. lung cancer, apathy, |
| | (sulfides). It is found in all soils and is emitted from volcanoes. | vertigo. Lung effects, including chronic bronchitis and |
| | Pure nickel is a hard, silvery-white metal that is combined with | reduced lung function, have been observed in workers |
| | other metals to form alloys. Some of the metals that nickel | who breathed large amounts of nickel |
| | can be alloyed with are iron, copper, chromium, and zinc. | |
| | These alloys are used in the making of metal coins and | |
| | jewelry and in industry for making metal items. Nickel | |
| | compounds are also used for nickel plating, to color ceramics | |
| | to make some batteries, and as catalysts that increase the | |
| | rate of chemical reactions. Nickel and its compound have no | |
| | characteristic odor or taste. Nickel is required to maintain | |
| | health in animals. A small amount of nickel is probably | |
| | essential for humans, although a lack of nickel has not been | |
| | found to affect the health of humans. Small nickel particles in | |
| | the air settle to the ground or are taken out of the air in rain. | |
| | Much of the nickel in the environment is found with soil and | |
| | sediments because nickel attaches to particles that contain | |
| | iron or manganese, which are often present in soil and | |
| | sediments. Nickel does not appear to collect in fish, plants or | |
| | animals used for food. Major sources of exposure are: | |
| | tobacco smoke, auto exhaust, fertilizers, super phosphate, | |
| | food processing, hydrogenated-fats-oils, industrial waste, | |
| | stainless steel cookware, testing of nuclear devices, tobacco | |
| | smoke, baking powder, combustion of fuel oil, dental work | |
| | and bridges. | |
| Chromium | Chromium is used in manufacturing chrome- | Breathing contaminated workplace air (stainless steel |
| | nickel-steel alloys (stainless steel) and other alloys, bricks in | welding, chromate or chrome pigment production, |
| | furnaces, and dyes and pigments, for greatly increasing | chrome plating, leather tanning). Handling or breathing |
| | resistance and durability of metals and chrome plating, | sawdust from chromium treated wood. Breathing |
| | leather tanning, and wood preserving. Manufacturing, | contaminated air, or ingesting water, or food from soil |
| | disposal of products or chemicals containing chromium, or | near waste sites or industries that use chromium. Very |
| | fossil fuel burning release chromium to the air, soil, and water. | small amounts of chromium (III) are in everyday foods. |
| | | All forms of chromium can be toxic at high levels, but |
| | | chromium (VI) is more toxic than chromium (III). Acute |
| | | toxic effects occur when breathing very high levels of |
| | | chromium (VI) in air that can damage and irritate your |
| | | nose, lungs, stomach, and intestines. People who are |
| | | allergic to chromium may also have asthma attacks |
| | | after breathing high levels of either chromium (VI) or |
| | | (III). Long term exposures to high or moderate levels of |
| | | chromium (VI) cause damage to the nose (bleeding, |
| | | itching, sores) and lungs, and can increase your risk of |
| | | non-cancer lung diseases. Ingesting very large |
| | | amounts of chromium can cause stomach upsets and |
| | | ulcers, convulsions, kidney and liver damage, and even |
| | | death. We don't know if chromium harms the fetus or |
| | | our ability to reproduce. Mice that ingested large |
| | | amounts of chromium had reproductive problems and |
| | | offspring with birth defects. Skin contact with liquids or |
| | | solids containing chromium (VI) may lead to skin |
| | | ulcers. Some people have allergic reaction including |
| | | severe redness and swelling. The Department of |
| | | Health and Human Services has determined that |
| | | certain chromium (VI) compounds are known |
| | | carcinogens. This is based on increased lung cancer in |
| | | some workers who were exposed to chromium. Animal |
| | | studies also indicate chromium (VI) is a carcinogen. |
| | | We do not have enough data to determine if chromium |
| | | (0) or chromium (III) are carcinogens. Chromium can |
| | | be measured in the hair, urine, serum, red blood cells, |
| | | and whole blood. Tests more useful for high-level |
| | | exposed people. Exact levels of exposure or how these |
| | | levels will affect health cannot be inferred from tests. |
| | | Chromium allergy may be detected by skin patch test. |

The following PSC/PGH from certain plant bud (or sap) sources have been found to successfully remove certain toxins:

Black Poplar—Populus Nigra (buds). A liquid extract derived from the buds of black poplar administered according to the method of the invention removes all heavy metals and chlorinated solvent, nitrates, 1,4-dioxan (widely used as a solvent in paints, varnishes, lacquers, cosmetics and deodorants). It also removes petroleum and its hydrocarbons.

Grape Vine—Vitis Vinifera (buds). A liquid extract derived from the buds of grape vine administered according to the method of the invention removes lead.

Mountain Pine—Pinus Montana (buds). A liquid extract derived from the buds of mountain pine administered according to the method of the invention removes all heavy metals

White Willow—Salix Alba (buds). A liquid extract derived from the buds of white willow administered according to the method of the invention removes mercury and other heavy metals.

European Alder—Alnus Glutinosa (buds) A liquid extract derived from the buds of European alder administered according to the method of the invention removes heavy metals, aluminum, and pesticides.

Silver Birch—Betula Verrucosa (sap). A liquid extract derived from the sap of silver birch has unique properties including the unparalleled ability, when administered according to the method of the invention, to assist in the discharge of undesired waste matter from the human body. A silver birch extract is particularly suited to be combined with other extract from different buds, depending on the overall test result of a particular patient.

### Clinical Observations and Methods

After patients (either adult or pediatric patients) exhibited symptoms characteristic for a certain toxin and was positively diagnosed for been afflicted by that toxin a concentrated PSC/PGH therapy was designed and administered for at least six months. The PSC/PGH therapy agents include 31 units of concentrated buds, 15 units of concentrated young shoots, 3 units of concentrated barks (in the embryonic stage), 3 units of concentrated rootlets, 1 unit of seed in the germinated embryonic stage 1 unit of concentrated male catkins flower, 1 unit of flower, and 1 unit of concentrated tree sap (The concentrated extract is commercially available form Herbal Gem, Vielsalm, Belgium). As described above, six specific PSC/PGH therapy agents eliminate various toxins. Bud extracts from birch trees are known to be active in cleansing the kidneys of waste and of correcting kidney function. When using birch, which has a diuretic effect, potassium, levels do not have to be checked since all birch contains 30% potassium therefore making it an ideal diuretic.

### Test Results:

Some 50 patients of all ages had positive urine screens for various heavy metals. Adults underwent a therapy which included 5 drops of a specific bud extract taken three time a day for each heavy metal for which they tested positive. Patients received drops for a minimum of six months. The dosage for children was 3 drops per heavy metal three times a day. The therapy proved successful at improving symptoms of heavy metal toxicity, as confirmed by achieving negative urine screens in 100% of patients. Most patients required therapy for a minimum of 6 months, although some patients needed to continue therapy for up to one year to further improve associated symptoms.

Among children with ADHD and autism, there has been significant improvement in health and mental IQ, cognitive functions, inattentiveness and hyperactive-compulsive behavior. These children are no longer showing symptoms of autism or of ADHD. The results are surprising and unprecedented.

### Testing for Heavy Metals

### Arsenic

Random urine <35 μg/g creatinine (Not provoked with a chelator)

Whole blood (short half-life in blood) <23 μg/L (Urine is more reliable for long-term exposure)

### Cadmium

Random urine <2.0 μg/g creatinine (Not provoked with a chelator)

### Lead

Random urine <150 μg/g creatinine (Not provoked with a chelator)

Whole blood <19 μg/dL

OSHA upper limit <40 μg/dL (upper limit for industrial exposure)

Provoked urine <600 mcg total in 24 hour urine collected immediately after 2.0 gm oral DMSA or 1.0 gm IV EDTA

### Mercury

Random urine <5 μg/g creatinine (Not provoked with a chelator)

Occupational limit in urine of exposed workers <35 μg/g creatinine (Not provoked with a chelator)

Whole blood <8.0 μg/L Occupational limit in exposed workers <15.0 μg/L

Hair <15 μg/g (μg/g=ppm)

Hair mercury is considered a valid test if properly performed. The recent Seychelles Island study showed that hair mercury below 15 μg/g (mean 6.9 ppm, SD 4.5 ppm) did not cause any problems in pregnant mothers or their newborn infants, who were followed with extensive neurological testing for many years from birth onwards. The diet contained ocean fish 12 meals per week. The fish contained the same amount of methyl mercury as found elsewhere in the world.

The World Health Organization's guidelines maintain that the lowest level that could possibly be harmful to humans is 5 parts per million (ppm). This level is based on scientific results from the 1960s that placed the level at which risk begins at 50 ppm for most people; WHO then applied a safety factor of 10, deciding that a level of 5 or less is safe for even the most vulnerable populations. Now the University of Rochester team has conducted an extensive study in the Seychelles Islands of the most sensitive population—young children—where the average level is about 7 ppm, about 10 times the level of the U.S. population. The scientists found no harm from mercury at levels up to 15 ppm, nearly twice the average Seychelles level and about 20 times higher than the average U.S. level. Environmental exposure: <8.0 μg/L, individuals consuming large quantities of seafood may have values as high as 200.0 μg/L. Occupational exposure: BEI®: inorganic mercury (sampling time is end of shift at end of work week): <15.0 μg/L.

Acute and chronic mercury poisoning affects the kidneys, central nervous system, and the gastrointestinal tract. The three telltale symptoms of mercury poisoning are impaired articulation, irregularity of muscular action, and constricted visual fields. Mercury poisoning through chronic exposure to metallic and inorganic forms of mercury generally produce nervousness, lassitude, tremor, and mucous membrane irritation. Inorganic mercury poisoning is associated primarily with peripheral effects, including gastroenteritis and tubular nephritis, whereas organic compounds predominantly affect the central nervous system (CNS), which may be severe and irreversible.

Chronic inorganic mercury poisoning is an occupational disease of smelters, mercury miners, gilders, and factory workers. Inhalation of mercury vapors may lead to pneumonitis, cough, fever, and other pulmonary symptoms. The most reliable way to measure exposure to inorganic mercury is to measure urinary mercury levels. Correlation between urine levels and symptoms is poor, however.

The most common non industrial source of mercury poisoning is the consumption of methyl mercury-contaminated fish. Organic mercury poisoning is best detected in whole blood, as this form of mercury is located mainly in the RBCs. Organic mercury poisoning may develop quickly and is usually a more serious disease.

BEI® are reference values intended as guidelines for evaluation of occupational exposure. BEI® represent biological levels of chemicals that correspond to workers with inhalation exposure equivalent to the threshold limit value (TLV®) of the chemicals. TLV®s refer to the airborne concentrations of substances and represent conditions under which it is believed that nearly all workers may be repeatedly exposed, day after day, without adverse health effects.

### Test Result of 7 Patients Treated with PSC/PGH Chelation

| | Positive 24 hour | Test result | |
|---|---|---|---|
| Patients age and | Urine Test | after 6 | References |
| gender | Results before | months | Range |
| Male 13 yrs old | Arsenic 275 | Arsenic <10 | <80 μg/L or less |
| Female 35 yrs old | Mercury 160 | Mercury 2 | <20 μg/L or less |
| | Arsenic 280 | Arsenic <10 | <80 μg/L or less |
| Male 42 yrs old | Cadmium 25 | Cadmium 0.5 | <5 μg/L or less |
| Male 12 yrs | Lead 120 | Lead <10 | <5 μg/L or less |
| Female 56 yrs old | Arsenic 350 | Arsenic 20.8 | <80 μg/L or less |
| Female 62 yrs old | Arsenic 250 | Arsenic <10 | <80 μg/L or less |
| | Mercury 180 | Mercury <10 | <20 μg/L or less |
| Male 70 yrs old | Arsenic 280 | Arsenic <10 | <80 μg/L or less |

None of the patients showed any essential mineral depletion.

Concentrated PSC/PGH Therapy vs. Diluted 1DH PSC/PGH Therapy

The use of concentrated PSC/PGH therapy holds advantages over standard PSC/PGH therapy. A problem with Diluted 1DH PSC/PGH therapy methods for chelation of heavy metals is the amount of alcohol that is ingested with this first decimal (1DH) dilution dosage, since the bud extract of PGH/PSC maintained in larger amount of alcohol. While concentrated PSC/PGH therapy requires only 5 drops three times per day, the requirements for the Diluted 1DH PSC/PGH therapy dosage requires 50 drops 3 times per day.

As metals and or toxins are removed from the body, some amount of regression or plateauing in the short-term is nearly inevitable, e.g., as their child reacts to the toxic metals being pulled through their body and brain.

Chelation, when done properly and for a long enough period of time, can produce dramatic improvement in patients.

It has been observed that chelation with concentrated PSC/PGH can be administered to patients of all ages.

Combined with the PSC/PGH are guidelines of making sure that the causes that need to be remedied are also minimized if not eliminated. Thus, the patient is instructed on the following issues: (1) Bring the toxin load down, (2) Remove casein (dairy) and gluten (wheat) from the diet. (3) Remove other food allergens from the diet. (4) Get rid of bad food habits and sugar. (5) Remove conventional household cleaners. (6) Add a HEPA air filter to your child's room. (7) Cook to keep toxins down. Choose organic items. Cook only in stainless steel pots and pens (non-stick pans have aluminum) and avoid cooking on or with aluminum foil as aluminum is a neuro-toxin itself and synergistically toxic with mercury. Avoid microwaving and storing food in plastic containers (plastic compounds leech into the food), (8) Use fluoride-free toothpaste (9) Avoid flame-retardant clothing. (10) Limit the use of pesticides, insecticides, and chemical fertilizers. (11) If a child needs dental work, do not use “silver” fillings, as they contain mercury. (12) If a child needs a vaccination, ensure they are Thimerosal-free. (13) Reduce fish intake.

Thus, while there have been shown and described and pointed out fundamental novel features of the invention as applied to a method of use thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Substitutions of elements from one described embodiment to another are also fully intended and contemplated. It is also to be understood that the drawings are not necessarily drawn to scale but that they are merely conceptual in nature. It is the intention, therefore, to be limited only as indicated by the scope of the claims appended hereto.

What is claimed is:

1. A method of using a plant stem cell/plant growth hormone-chelation preparation including plant stem cells/plant growth hormones as a chelation agent.

2. The method of using a plant stem cell/plant growth hormone chelation preparation, comprising the steps of
determining from medical testing the toxic condition of the patient,
selecting a plant stem cell/plant growth hormone plant extract concentrate from buds of at least one plant having nutritional and medicinal constituents,
providing for oral administration of the plant stem cell/plant growth hormone plant extract an amount of about 3-15 drops, diluted in about half a cup of water, three times a day,
continue the oral administration for about 3 to 6 months or until follow up testing provides a negative result.

3. The method of using a plant stem cell/plant growth hormone chelation preparation, comprising the steps of
determining from medical testing the toxic condition of the patient,
selecting a combination of plant stem cell/plant growth hormone plant extract concentrate from at least two plants having nutritional and medicinal constituents,
providing for oral administration the combination of the plant stem cell/plant growth hormone plant extracts in an amount of about 3-15 drops, diluted in about half a cup of water, three times a day
continue the oral administration for about 3 to 6 months or until follow up testing provides a negative result.

4. The method of claim 2, wherein the plant stem cells/plant growth hormones concentrate are prepared from the buds of at least one of black poplar, grape vine, white willow, European elder and silver birch concentrate, wherein the concentrate is prepared by maceration process using at least water, alcohol and glycerine.

5. The method of claim 3, wherein the plant stem cells/plant growth hormones concentrates are prepared from the buds of at least two plants selected from black poplar, grape vine, white willow, European elder and silver birch concentrate, wherein the concentrate is prepared by maceration process using at least water, alcohol and glycerine.

6. The method of claim 2, wherein the plant stem cells/plant growth hormones concentrate is prepared from the buds, young shoots or rootlets of any of black poplar, grape vine, white willow, European elder and silver birch concentrate, wherein the concentrate is prepared by maceration process using at least water, alcohol and glycerine.

7. The method of claim 2, wherein the plant stem cells/plant growth hormones of the buds are derived from a grape vine concentrate and is prepared by maceration process using at least water, alcohol and glycerine.

8. The method of claim 2, wherein the plant stem cells/plant growth hormones of the buds are derived from mountain pine concentrate and is prepared by maceration process using at least water, alcohol and glycerine.

9. The method of claim 2, wherein the plant stem cells/plant growth hormones of the buds are derived from a white willow concentrate and is prepared by maceration process using at least water, alcohol and glycerine.

10. The method of claim 2, wherein the plant stem cells/plant growth hormones of the buds are derived from a European elder concentrate and is prepared by maceration process using at least water, alcohol and glycerine.

11. The method of claim 2, wherein the plant stem cells/plant growth hormones of the buds are derived from a silver birch concentrate and is prepared by maceration process using at least water, alcohol and glycerine.

12. The method of claim 2, wherein the plant stem cells/plant growth hormones of the buds are derived from at least two of black poplar, grape vine, mountain pine, silver birch, white willow, European Elder.

13. A method of treating heavy metal toxicity and associated symptoms comprising administering plant stem cell/plant growth hormone preparation orally, the plant stem cell/plant growth hormone preparation is an extract from at least one of black poplar, grape vine, white willow, European alder and silver birch concentrate.

14. The method of treating heavy metal toxicity and associated symptoms of claim 2, wherein the extract is prepared by maceration process using at least water, alcohol and glycerin.

15. A method of treating toxicity and associated symptoms comprising administering plant stem cell/plant growth hormone preparation orally, the plant stem cell/plant growth hormone preparation is an extract from at least one of black poplar, grape vine, white willow, European elder and silver birch concentrate.

16. The method of treating toxicity and associated symptoms of claim 15, wherein the extract is prepared by maceration process using at least water, alcohol and glycerin.

17. A biologically active extract form at least two of the buds from the group of black poplar, grape vine, white willow, European elder and silver birch.

18. The biologically active extract according to claim 17, wherein the extract is prepared by maceration process using at least water, alcohol and glycerin and diluting the extract with water.
